(19) United States
(12) Patent Application Publication
(10) Pub. No.: US 2004/0077951 A1
(43) Pub. Date: Apr. 22, 2004

(54) Apparatus and methods of detection of radiation injury using optical spectroscopy

(76) Inventors: Wei-Chiang Lin, Nashville, TN (US); Steven A. Toms, Lake Osewego, OR (US); Anita Mahadevan-Jansen, Nashville, TN (US); Paul J. Phillips, Knoxville, TN (US); Mahlon Johnson, Nashville, TX (US); Robert J. Weil, Nashville, TN (US)

Correspondence Address: MERCHANT & GOULD PC, P.O. BOX 2903, MINNEAPOLIS, MN, 55402-0903, US

(21) Appl. No.: 10/613,225

(22) Filed: Jul. 3, 2003

Related U.S. Application Data
(60) Provisional application No. 60/394,217, filed on Jul. 5, 2002.

(51) Int. Cl. A61B 6/00

(52) U.S. Cl. 600/476

(57) ABSTRACT

An apparatus and method for detecting radiation damage in an area of brain tissues, where the area of brain tissues has at least a first region containing brain tissues damaged from radiation exposure and a second region containing no brain tissues damaged from radiation exposure. In one embodiment, the method includes the steps of illuminating in vivo the area of brain tissues with a coherent light at an incident wavelength, λ₀, between 330 nm and 360 nm, collecting electromagnetic emission returned from the illuminated brain tissues, and identifying a first peak of intensity of the collected electromagnetic emission at a first wavelength, λ₁, and a second peak of intensity of the collected electromagnetic emission at a second wavelength, λ₂, wherein λ₀, λ₁, and λ₂ satisfy the following relationship of λ₁>λ₂>λ₀. The method further includes the step of locating the first region containing brain tissues damaged from radiation exposure as the region of brain tissues where the first peak of intensity of the collected electromagnetic emission is corresponding to.

### CROSS-REFERENCE TO RELATED APPLICATION

This application claims priority to U.S. Provisional Application Serial No. 60/394,217, which was filed on Jul. 5, 2002, in the United States Patent and Trademark Office, and is hereby incorporated herein by reference in its entirety.

The present invention was made in part with Government support through a grant awarded by the National Institutes of Health. The United States Government may have certain rights to this invention pursuant to the grant.

Some references, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference was individually incorporated by reference. In terms of notation, hereinafter, “[n]” represents the nth reference cited in the reference list. For example, [13] represents the 13th reference cited in the reference list, namely, Lin W-C, Toms S A, Johnson M, et al: In vivo brain tumor demarcation using optical spectroscopy, Photochem Photobio, 2001, 73:396-402.

### FIELD OF THE INVENTION

The present invention generally relates to detection of radiation damage in an area of brain tissues, and in particular to the utilization of optical spectroscopy, such as fluorescence spectroscopy, in vivo to detect radiation damage in an area of brain tissues.

### BACKGROUND OF THE INVENTION

Radiation therapy is commonly used in conjunction with surgical resection to treat patients with brain tumors in order to improve patient's long-term, disease-free survival. Unfortunately, ionizing radiation has limited tissue specificity and damages both normal and neoplastic brain tissues. The most serious form of central nervous system (CNS) injury is radiation necrosis, the most severe delayed adverse effect of head and brain irradiation. This reaction usually occurs from 6 to 24 months after therapeutic irradiation and in up to 5% of those who receive more than 6000 cGy in conventional fractions of 200 cGy/day [1, 2]. The pathophysiology of radiation damage to the CNS is characterized by transient demyelination, the proliferation of oligodendrocytes, and changes in the blood-brain barrier [3, 4], which corresponds to the normal brain components, endothelial cells, oligodendrocytes with the most rapid tumor rates. Histopathologically, cerebral radiation necrosis is characterized by endothelial proliferation, extensive gliosis and neuronal loss, and is most prominent in the subcortical white matter [5].

After the initiation of radiation therapy, imaging studies such as computed tomography (CT) and magnetic resonance imaging (MRI) are used in the post-treatment surveillance of brain tumors. It is common to see several types of radiation-induced lesions including hypodensities on CT, increased T2-weighted signal abnormalities on MRI, or evolving areas of contrast enhancement [5]. The most important clinical distinction in these patients is the identification of new contrast-enhancing masses as either recurrent tumor or radiation necrosis, or a combination of tumor and radiation damage. Unfortunately, with CT and MRI, it is often impossible to differentiate these two entities.

A number of alternative imaging techniques have been employed in order to non-invasively determine whether the areas of enhancement seen on MRI and CT correspond to recurrent tumor or radiation necrosis. Nuclear medicine imaging techniques such as single photon emission tomography (SPECT) and positron emission tomography (PET) have been the subject of numerous studies. SPECT scanning relies upon the alterations in regional blood flow (99 mTc DPTA) or the blood brain barrier (SPECT 201-T1 chloride) [6, 7] in radionecrotic areas to predict radiation damage. PET scanning, on the other hands, relies upon metabolites such as glucose or methionine to determine the presence of tumor. Although these methods continue to be avidly studied, the positively predictive value of these techniques has ranged from 60 to 90 percent [8, 9]. As a result of the shortcomings of nuclear medicine imaging, several MR techniques, including dynamic susceptibility MRI [10] and MR spectroscopy (MRS) have been assessed. Of these techniques, proton (¹H) RS seems the most promising. Predictive values of between 70 and 96 percent in the diagnosis of radiation necrosis have been recently reported [11, 12].

Despite these encouraging attempts at non-invasive differentiation of cerebral radionecrosis from recurrent tumor, tissue diagnosis remains the gold standard in the art upon which most treatment decisions are made. However, even stereotactic biopsy of enhancing lesions identified on MRI may be inaccurate due to sampling error. Unless a craniotomy is performed, the entire region of abnormality resected, and the tissue subject to detailed histopathologic analysis, small rests of viable tumor within areas of otherwise confluent radionecrosis may remain undiagnosed. As any viable tumor cells may lead to treatment failure and death, the discovery of viable tumor cells may radically alter clinical decision-making. Therefore, any improvements in techniques to distinguish radiation changes from recurrent tumor cells should aid the clinician in more accurate diagnosis.

Optical spectroscopy, such as fluorescence spectroscopy, has been shown capable of detecting subtle changes in both biochemical compositions and morphological characteristics of tissues associated with the progression of disease in near real-time, where these differences can be used to detect tissue abnormalities and ultimately lead to optical tissue discrimination. Optical spectroscopy has been successfully applied to detect disorders of various organs such as cervix, skin, and the like [13, 14, 15] both in vitro and in vivo. Several commercial systems are currently available for clinical diagnosis in the bronchus, cervix, etc. However, relatively few studies have addressed the diagnostic potential of optical spectroscopy in brain tumors. Particularly, fluorescence characteristics of radiation-injured brain tissues have not yet been developed.

Thus, there still is a need in the art to address the aforementioned deficiencies and inadequacies.

### SUMMARY OF THE INVENTION

The present invention, in one aspect, relates to a method for detecting radiation damage in an area of brain tissues, where the area of brain tissues has at least a first region containing brain tissues damaged from radiation exposure and a second region containing no brain tissues damaged from radiation exposure. The first region of the area of brain tissues comprises tumor. The second region of the area of brain tissues comprises normal brain tissues, and it may further comprise tumor.

In one embodiment, the method includes the steps of illuminating in vivo the area of brain tissues with a coherent light at an incident wavelength, λ₀, between 330 nm and 360 nm, collecting electromagnetic emission returned from the illuminated brain tissues, and identifying a first peak of intensity of the collected electromagnetic emission at a first wavelength, λ₁, and a second peak of intensity of the collected electromagnetic emission at a second wavelength, λ₂, wherein λ₀, λ₁, and λ₂ satisfy the following relationship of λ₁>λ₂>λ₀. The method further includes the steps of locating the first region containing brain tissues damaged from radiation exposure as the region of brain tissues where the first peak of intensity of the collected electromagnetic emission is corresponding to.

Additionally, the incident wavelength, λ₀, of the coherent light is emitted from a laser light source and is substantially at around 337 nm. The first wavelength, λ₁, of the collected electromagnetic emission is substantially at around 500 nm. And the second wavelength, λ₂, of the collected electromagnetic emission is substantially at around 460 nm.

In another aspect, the present invention relates to an apparatus for detecting radiation damage in an area of brain tissues, where the area of brain tissues has at least a first region containing brain tissues damaged from radiation exposure and a second region containing no brain tissues damaged from radiation exposure.

In one embodiment of the present invention, the apparatus includes means for illuminating in vivo the area of brain tissues with a coherent light at an incident wavelength, λ₀, between 330 nm and 360 nm, means for collecting electromagnetic emission returned from the illuminated brain tissues, means for identifying a first peak of intensity of the collected electromagnetic emission at a first wavelength, λ₁, and a second peak of intensity of the collected electromagnetic emission at a second wavelength, λ₂, wherein λ₀, λ₁, and λ₂ satisfy the following relationship of λ₁>λ₂>λ₀, and means for locating the first region containing brain tissues damaged from radiation exposure as the region of brain tissues where the first peak of intensity of the collected electromagnetic emission is corresponding to.

Furthermore, the means for illuminating has a laser light source. The means for collecting includes a fiber optical probe coupled with the laser light source so as to deliver in vivo the laser light to an area of brain tissues proximal a working end of the probe. The means for collecting further includes a spectroscope coupled with the fiber optical probe so as to receive from the working end of the probe, fluorescent light emitted from the area in response to illumination by the coherent light. And the means for identifying comprises a system controller operatively coupled with the spectroscope.

In a further aspect, the present invention relates to an apparatus for detecting radiation damage in an area of brain tissues, where the area of brain tissues has at least a first region containing brain tissues damaged from radiation exposure and a second region containing no brain tissues damaged from radiation exposure.

In one embodiment, the apparatus has a laser light source emitting a coherent light with an incident wavelength, λ₀, between 330 nm and 360 nm, and a fiber optical probe coupled with the laser light source so as to deliver in vivo the laser light to an area of brain tissues proximal a working end of the probe, and a spectroscope coupled with the fiber optical probe so as to receive from the working end of the probe, fluorescent light emitted from the area in response to illumination by the coherent light, and a system controller operatively coupled with the spectroscope and configured to generate a spectrum having a first peak of intensity of the fluorescent light at a first wavelength, λ₁, and a second peak of intensity of the fluorescent light at a second wavelength, λ₂, wherein λ₀, λ₁, λ₂ satisfy the following relationship of λ₁>λ₂>λ₀.

In operation, the first region containing brain tissues damaged from radiation exposure is corresponding to the region of brain tissues where the first peak of intensity of the fluorescent light at the first wavelength λ₁ is emitted, where the first wavelength, λ₁, is substantially at around 500 nm.

In yet another aspect, the present invention relates to a method for detecting radiation damage in an area of tissues associated with a living subject, wherein the area of tissues has at least a first region containing tissues damaged from radiation exposure and a second region containing no tissues damaged from radiation exposure. The living subject can be a human being or an animal. The area of tissues can be any part of tissues associated with a living human being or an animal.

In one embodiment, the method includes the steps of illuminating the area of tissues with a coherent light at an incident wavelength, λ₀, between 330 nm and 360 nm, and collecting electromagnetic emission returned from the illuminated tissues. The method further includes the steps of identifying a first peak of intensity of the collected electromagnetic emission at a first wavelength, λ₁, and a second peak of intensity of the collected electromagnetic emission at a second wavelength, λ₂, wherein λ₀, λ₁, and λ₂ satisfy the following relationship of λ₁>λ₂>λ₀, and locating the first region containing tissues damaged from radiation exposure as the region of tissues where the first peak of intensity of the collected electromagnetic emission is corresponding to. The method can be practiced in vivo or in vitro.

The incident wavelength, λ₀, of the coherent light is substantially at around 337 nm, wherein the coherent light is emitted from a laser light source. The first wavelength, λ₁, of the collected electromagnetic emission is substantially at around 500 nm. And the second wavelength, λ₂, of the collected electromagnetic emission is substantially at around 460 nm.

In a further aspect, the present invention relates to an apparatus for detecting radiation damage in an area of tissues associated with a living subject, wherein the area of tissues has at least a first region containing tissues damaged from radiation exposure and a second region containing no tissues damaged from radiation exposure. The living subject can be a human being or an animal. The apparatus can be utilized to practice the present invention in vivo or in vitro.

In one embodiment, the apparatus includes means for illuminating the area of tissues with a coherent light at an incident wavelength, λ₀, between 330 nm and 360 nm, and means for collecting electromagnetic emission returned from the illuminated tissues. The apparatus further includes means for identifying a first peak of intensity of the collected electromagnetic emission at a first wavelength, λ₁, and a second peak of intensity of the collected electromagnetic emission at a second wavelength, λ₂, wherein λ₀, λ₁, and λ₂ satisfy the following relationship of λ₁>λ₂>λ₀, and means for locating the first region containing tissues damaged from radiation exposure as, the region of tissues where the first peak of intensity of the collected electromagnetic emission is corresponding to.

The incident wavelength, λ₀, of the coherent light is substantially at around 337 nm. In one embodiment, the means for illuminating comprises a laser light source, and the means for collecting comprises a fiber optical probe coupled with the laser light source so as to deliver in vivo the laser light to an area of tissues proximal a working end of the probe. The means for collecting further comprises a spectroscope coupled with the fiber optical probe so as to receive from the working end of the probe, fluorescent light emitted from the area in response to illumination by the coherent light. Furthermore, the means for identifying comprises a system controller operatively coupled with the spectroscope. The first wavelength, λ₁, of the collected electromagnetic emission is substantially at around 500 nm. And the second wavelength, λ₂, of the collected electromagnetic emission is substantially at around 460 nm.

These and other aspects of the present invention will become apparent from the following description of the preferred embodiment taken in conjunction with the following drawings, although variations and modifications therein may be affected without departing from the spirit and scope of the novel concepts of the disclosure.

### BRIEF DESCRIPTION OF THE DRAWINGS

FIG. 1 is a schematic for in vivo detecting radiation damages in brain tissues of a patient.

FIG. 2 is a block diagram showing a flowchart for detecting radiation damage in brain tissues according to one embodiment of the present invention.

FIG. 3 is a block diagram of an apparatus for detecting radiation damage in brain tissues according to one embodiment of the present invention.

FIG. 4a shows MRI images of brain tissues of a selected patient.

FIG. 4b shows PET images of brain tissues of the selected patient in FIG. 4a.

FIG. 4c shows fluorescence spectra from brain tissues of the selected patient in FIG. 4a, where curve 401 (the open diamonds) represents data from an area of radionecrotic tissues mixed with tumor cells, curve 403 (the solid squares) represents data from an area of radionecrosis, and curve 405 (the open circles) represents data from an area of normal gray matter, respectively.

FIG. 4d shows diffuse reflectance spectra from brain tissues of the selected patient in FIG. 4a, where curve 411 (the open diamonds) represents data from an area of radionecrotic tissues mixed with tumor cells, curve 413 (the solid squares) represents data from an area of radionecrosis, and curve 415 (the open circles) represents data from an area of' normal gray matter, respectively.

FIG. 5a shows MRI images of brain tissues of another selected patient.

FIG. 5b shows PET images of brain tissues of the selected patient in FIG. 5a.

FIG. 5c shows fluorescence spectra from brain tissues of the selected patient in FIG. 5a, where curve 501 (the solid squares) represents data from an area of tissues with infiltrating tumor cells, curve 503 (the open circles) represents data from an area of radionecrosis, curve 505 (the open diamonds) represents data from an area of solid tumor, curve 507 (the crosses) represents data from an area of normal white matter, and curve 509 (the solid triangles) from represents data an area of radionecrotic tissues with tumor cells, respectively.

FIG. 5d shows diffuse reflectance spectra from brain tissues of the selected patient in FIG. 5a, where curve 511 (the solid squares) represents data from an area of tissues with infiltrating tumor cells, curve 513 (the open circles) represents data from an area of radionecrosis, curve 515 (the open diamonds) represents data from an area of solid tumor, curve 517 (the crosses) represents data from an area of normal white matter, and curve 519 (the solid triangles) represents data from an area of radionecrotic tissues with tumor cells, respectively.

### DETAILED DESCRIPTION OF THE INVENTION

Various embodiments of the invention are now described in detail. Referring to the drawings, like numbers indicate like parts throughout the views. As used in the description herein and throughout the claims that follow, the meaning of “a,” “an,” and “the” includes plural reference unless the context clearly dictates otherwise. Also, as used in the description herein and throughout the claims that follow, the meaning of “in” includes “in” and “on” unless the context clearly dictates otherwise. Additionally, some terms used in this specification are more specifically defined below.

### DEFINITIONS

The terms used in this specification generally have their ordinary meanings in the art, within the context of the invention, and in the specific context where each term is used.

Certain terms that are used to describe the invention are discussed below, or elsewhere in the specification, to provide additional guidance to the practitioner in describing the apparatus and methods of the invention and how to make and use them. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification, including examples of any terms discussed herein, is illustrative only, and in no way limits the scope and meaning of the invention or of any exemplified term. Likewise, the invention is not limited to various embodiments given in this specification.

As used herein, “around”, “about” or “approximately” shall generally mean within 20 percent, preferably within 10 percent, and more preferably within 5 percent of a given value or range. Numerical quantities given herein are approximate, meaning that the term “around”, “about” or “approximately” can be inferred if not expressly stated.

As used herein, “radiation therapy” refers to the use of ionizing radiation to treat disease.

As used herein, “radiation” refers to waves or particles of energy emitted from radioactive sources as used in radiation therapy.

The term “radiation injury,” or “radiation damage,” as used herein, is synonym in the specification and refers to damage to tissues caused by exposure to radiation.

The term “region,” or “site,” as used herein, is synonym in the specification and indicates an area of brain tissues.

### OVERVIEW OF THE INVENTION

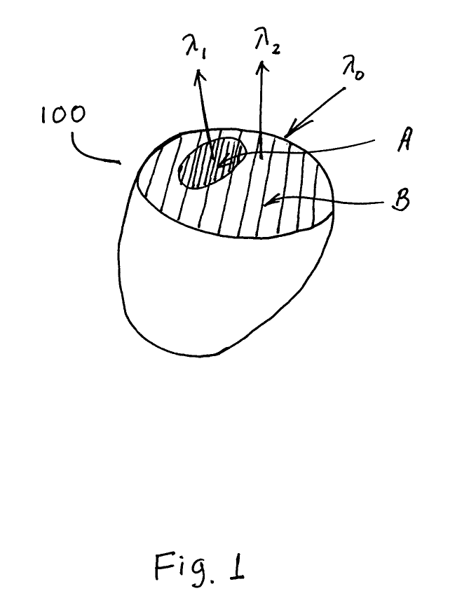

In one aspect, the present invention relates to a method for detecting radiation damage in an area of brain tissues of a subject such as a patient who has been subject to radiation therapy. As schematically shown in FIG. 1, the area of brain tissues 100 has at least a first region A containing brain tissues damaged from radiation exposure and a second region B containing no brain tissues damaged from radiation exposure. In other words, the brain tissues contain radiation damaged tumor in the first region A, and contain at least one of normal brain tissues and tumor in the second region B, respectively.

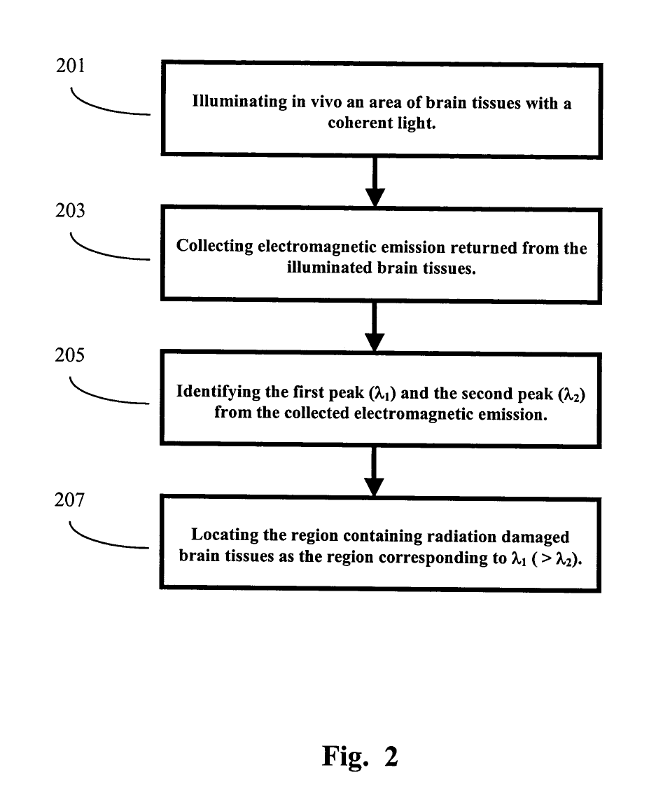

Referring in general to FIGS. 1 and 2, the method for detecting radiation damage according to one embodiment of the present invention includes the following steps: at step 201, the area of brain tissues is illuminated in vivo with a coherent light at an incident wavelength, λ₀, which is between 330 nm and 360 nm. In one example, the incident coherent light is emitted from a laser light source with a wavelength substantially at around 337 nm. At step 203, electromagnetic emission returned from the illuminated brain tissues is collected to form a spectrum or spectra. For instance, a portable, optical spectroscopic system can be employed to acquire in vivo fluorescence spectra. At step 205, a first peak of intensity of the collected electromagnetic emission spectra at a first wavelength, λ₁, and a second peak of intensity of the collected electromagnetic emission at a second wavelength, λ₂, are identified. The wavelengths λ₀, λ₁ and λ₂ satisfy the relationship of λ₁>λ₂>λ₀. Particularly, the first wavelength, λ₁, is substantially at around 500 nm and the second wavelength, λ₂, at around 460 nm. Once the first and second peaks are identified, at step 207, the first region containing brain tissues damaged from radiation exposure is located as the region of brain tissues where the first peak of intensity of the collected electromagnetic emission is corresponding to. In one embodiment, the area of the brain tissues is illuminated and the returned emission is processed sequentially such that when the collected emission spectra corresponding to an illuminated region of the brain tissues have a peak at wavelength substantially around 500 nm, that region of the brain tissues is identified as the first region, a region where the illuminated brain tissues are damaged from radiation exposure.

### EXAMPLES

### APPARATUS SETUP AND MEASUREMENT

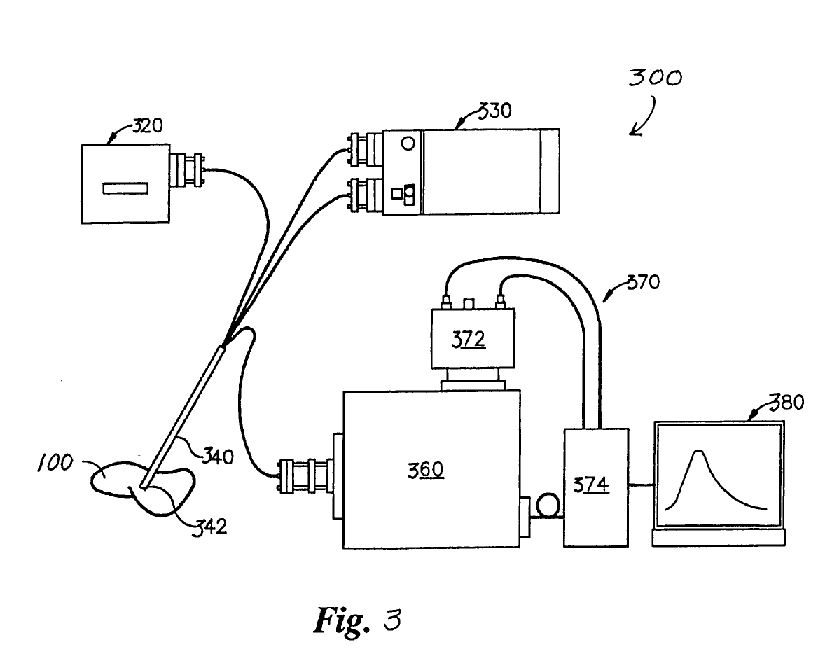

Referring now to FIG. 3, in one embodiment of the present invention, an apparatus 300 includes a source of laser light 330, a fiber optical probe 340 coupled with the source of laser light 330 so as to deliver the laser light to a working end 342 of the fiber optical probe 340, and a spectrograph or spectroscope 360 coupled with the fiber optical probe 340 so as to receive fluorescent light emitted from in vivo brain tissues 100 contacted by the working end 342 of the fiber optical probe 340 and providing a frequency spectrum of the fluorescent light. The apparatus 300 further has a frequency amplitude detector 370 in the form of a CCD camera 372 with a camera controller 374, and a processor 380 in the form of a PC coupled with the spectrograph 360 through the detector 370 and programmed to analyze the frequency spectrum of light carried from the working tip of the probe 340 to the spectrometer 360 to distinguish between light returned to the spectrograph 360 from radiation damaged brain tissues and from non-radiation damaged brain tissues.

Several types of laser light sources can be used to practice the present invention. In one embodiment, for example, a 337 nm high-pressure nitrogen laser 330 (e.g., a high pressure nitrogen dye laser from Oriel Corporation, Stratford, Conn.) is used as an excitation source for fluorescence measurements. Light delivery and collection can be achieved with a proper fiber optical probe such as a “Gaser” fiber optical probe 340 (Visionex, Inc., Atlanta, Ga.). This probe comprises a plurality of individual wave guides in the form of 300 micron core diameter glass fibers, wherein one delivers laser pulses to the detecting region of the brain tissues through the working end 342 of the probe 340, and one collects fluorescence emission induced by the incident laser light through the working end 342 of the probe 340. Prior to the measurement, the probe is gas-sterilized and then set up the operating field. The detailed structure and operation of the probe are exemplified in U.S. Pat. No. 6,377,841, which is incorporated herein by reference.

The collected fluorescent light is carried by the fiber optical probe 340 to the spectrograph 360 (e.g., a Triax 180 from Instruments S.A., Inc., Edison, N.J.) where it is dispersed and detected with detector 370, which is in the form of a CCD camera 372 with a camera controller 374, and projected onto a sensor array of a thermoelectrically cooled CCD camera 372 (e.g., a Spectra One from Instruments S.A., Inc., Edison, N.J.).

The wavelength dependent light intensity recorded by the CCD camera 372 is then read, stored and analyzed by the processor 380 in the form of a PC personal computer. The recorded spectral data can be analyzed according to a specific method or procedure as part of the present invention. In this procedure, the recorded optical spectra are processed to eliminate the artifacts in them, that is, baseline is first removed from the spectra, and calibration factors derived from a standard calibration procedure are then multiplied to the spectra to remove wavelength-dependent characteristics of the spectroscopic system 300 [16]. The processed spectra are then analyzed to identify spectral variations in line-shape (rather than intensity) induced by the radiation injuries of brain tissues. More specifically, when the fluorescence spectra emitted from an illuminated region of the brain tissues have a peak at wavelength substantially around 500 nm, this illuminated region of the brain tissues is then recognized as the first region, a region where the illuminated brain tissues are radiation damaged. Furthermore, when the florescence spectra from an illuminated region of the brain tissues have a peak at wavelength substantially around 460 nm, this illuminated region of the brain tissues is recognized as the second region, a region where the illuminated brain tissues are not radiation damaged.

Additionally, as shown in FIG. 3, the present invention can also be practiced to investigate diffuse reflectance spectra from an area of brain tissues by incorporating an optional source of broadband white light 320 (for example, a Fiber Lite, Model 180, Edmund Industrial Optics, Barrington, N.J.). However, as set forth below in more details, the diffuse reflectance spectroscopy in detecting radiation injuries of brain tissues is not as effective as the fluorescence spectroscopy according to the present invention.

### RESULTS AND IMPLICATIONS

In one embodiment of the present invention, fifteen brain tumor patients, who previously had undergone radiation therapy and underwent surgical resection for recurrent enhancing mass lesion, are selected and studied over a 6-month study period. Table 1 provides the medical history for each of the fifteen patients enrolled in the study. Each patient, or a subject of study, was assigned a number from 1 to 15 as his or her identification. For example, patient No. 3 corresponds to a patient who was 51 years old at the time of enrollment and had radiation necrosis/meningioma as shown in Table 1.

TABLE 1
Medical history of patients enrolled in the study.

| Patient | | | Fractionated | Interval | Stereotactic | Interval |
|---|---|---|---|---|---|---|
| No. | Age | Histology | XRT (Y/N) | (months) | XRT (Y/N) | (months) |
| 1 | 58 | GBM | Y | 0 | N | — |
| 2 | 25 | Recurrent GBM | Y | 5 | Y | 2 |
| 3 | 51 | Radiation | Y | 26 | Y | 5 |
| | | necrosis/ | | | | |
| | | meningioma | | | | |
| 4 | 46 | AA + XRT | Y | 7 | N | — |
| | | changes | | | | |
| 5 | 54 | Recurrent GBM + | Y | 17 | Y | 5 |
| | | XRT changes | | | | |
| 6 | 60 | Recurrent AA | Y | 21 | N | — |
| 7 | 53 | Recurrent AA | N | — | Y | 3 |
| 8 | 57 | Recurrent GBM | Y | 10 | N | — |
| 9 | 49 | Recurrent GBM | Y | 8 | N | — |
| 10 | 45 | Recurrent GBM | Y | 13 | N | — |
| 11 | 38 | Recurrent AA | Y | 14 | Y | 16 |
| 12 | 47 | Radiation | Y | 24 | Y | 6 |
| | | necrosis | | | | |
| 13 | 44 | Recurrent AO | N | — | Y | 60 |
| 14 | 39 | Recurrent GBM | Y | 9 | N | — |
| 15 | 54 | Recurrent GBM | Y | 5 | N | — |

An unique fluorescence spectral emission peak at wavelength around 500 nm was found in at least 13 of the 15 studied patients. Data from two representative patient studies are presented in detail below to illustrate the correlation between this unique fluorescence spectral characteristic and radiation injuries of brain tissues.

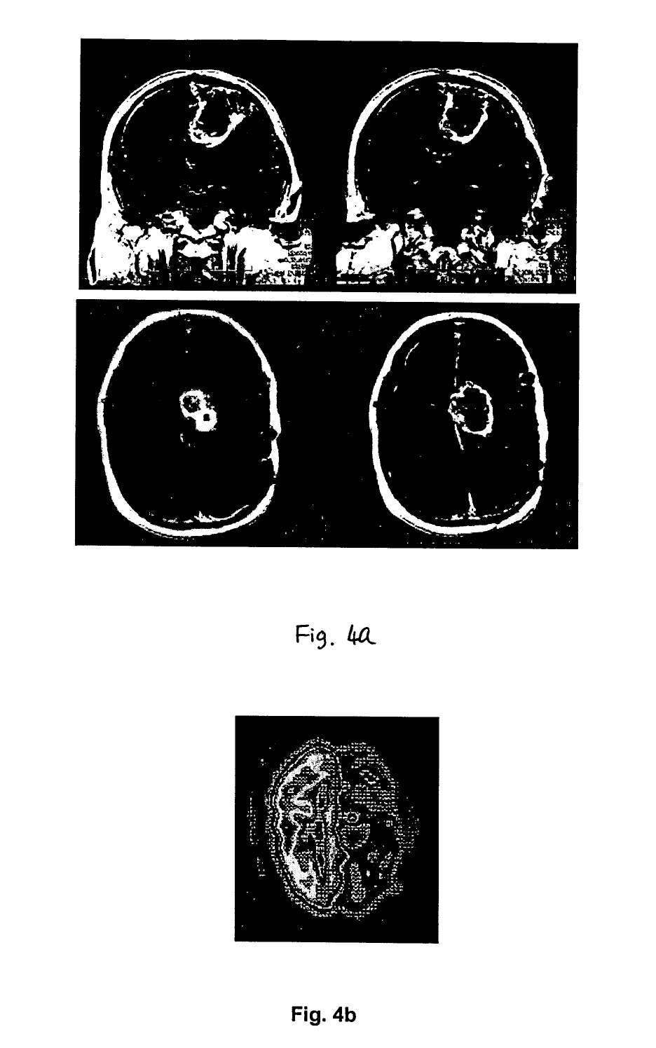

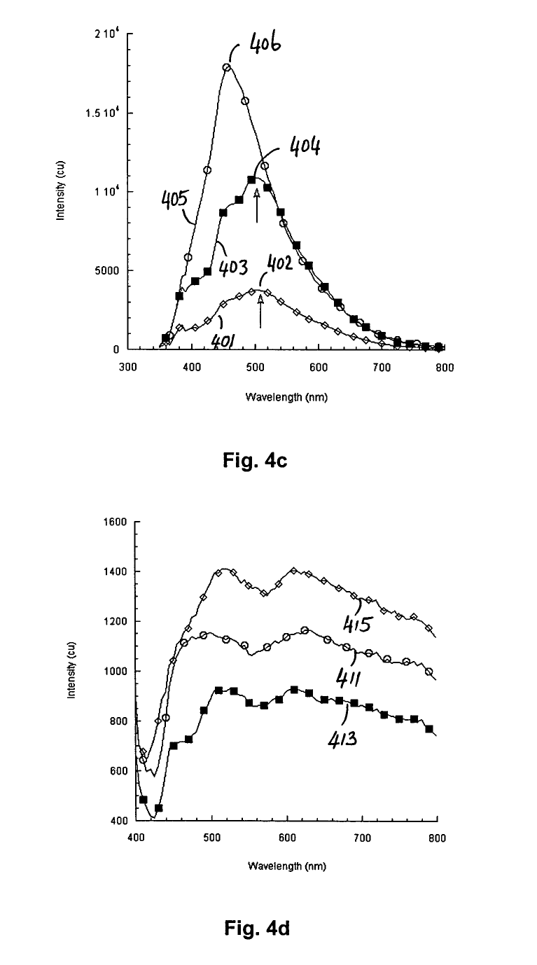

The data set shown in FIG. 4 is obtained from a patient, listed as patient No. 3 in Table 1, with a history of meningioma and multiple surgical resections and irradiation including postoperative external beam radiotherapy and Gamma Knife™ radiosurgery at an outside institution. MRI images six months after Gamma Knife™ radiosurgery, as displayed in FIG. 4a, showed vigorous enhancement in the areas of prior radiosurgery, suggestive of recurrent tumor. PET scanning image, as shown in FIG. 4b, showed central hypometabolism and a peripheral hypermetabolism interpreted by the nuclear medicine physicians as suggestive of recurrent tumor. Due to worsening mass effect and hemiparesis, a craniotomy was performed and optical spectral measurements were conducted. Five of the eight investigated tissue sites, located at the center as well as borders of the enhanced area, showed clear radiation-induced changes histologically. Small amounts of residual meningioma were found in two investigated sites.

Referring now to FIGS. 4c and 4d, fluorescence and diffuse reflectance spectra from three representative sites in this patient are shown, respectively. Correspondingly, there are three spectrum curves 401, 403, and 405 for an area of radionecrotic tissues mixed with tumor cells, an area of radionecrosis, and an area of a normal gray matter, respectively. As shown in FIG. 4c, an unique fluorescence peak 404 at wavelength substantially around 500 nm is identified in the fluorescence spectrum curve 403, which is corresponding to a representative site or an area that contains radiation damaged brain tissues as confirmed by histology. Additionally, a secondary, yet an unique peak 402 at wavelength substantially around 500 nm is identified in curve 401, which is corresponding to a representative site that contains radionecrotic tissues mixed with tumor cells. In comparison, curve 405 from a normal gray matter area has a fluorescence spectral peak 406 substantially at around 460 nm. Peak 402 and peak 404 can be termed as a first peak and peak 406 can be termed as a second peak. Diffuse reflectance spectra for the three representative sites, shown as curves 411, 413, and 415 in FIG. 4d, respectively, display no distinct spectral variations that could be correlated to histological changes compatible with radiation damage.

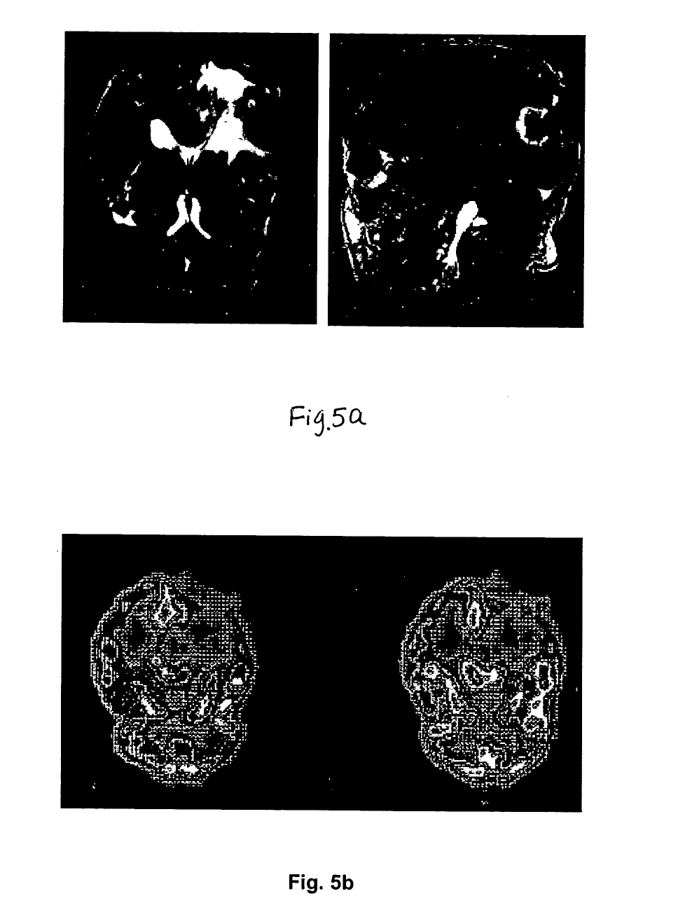

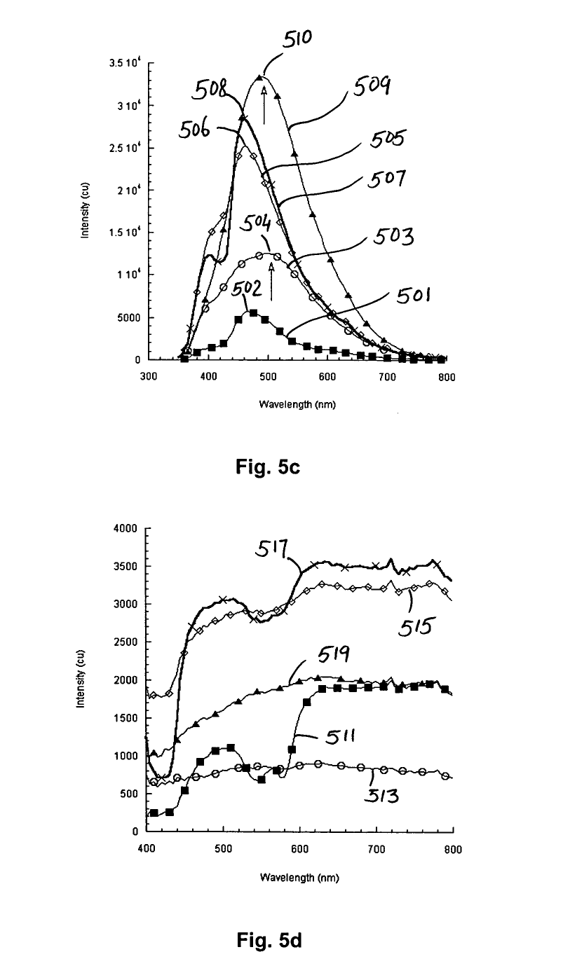

The second case illustration, shown in FIG. 5, is from a patient, listed as patient No. 5 in Table 1, with recurrent glioblastoma multiforme (GBM) who had undergone two prior craniotomies, fractioned external beam radiotherapy, as well as stereotactic radiosurgery prior to the clinical study performed according to the present invention. Preoperative MRI images of patient No. 5, as shown in FIG. 5a, displays drastic enhancement in several areas, which suggests recurrent tumors or radionecrosis. PET scanning images of patient No. 5, as given in FIG. 5b, shows areas of low metabolism as well as mild peripheral hypermetabolism. The patient had progressive neurologic deterioration that prompted additional resection.

Histological examinations of tissues from the third operation on patient No. 5 show both tumor cells and radiation injuries. Fluorescence spectra measured from five investigated sites are observed. Correspondingly, as shown in FIG. 5c, there are five spectrum curves 501, 503, 505, 507, and 509 for an area of infiltrating tumor cells, an area of radionecrosis, an area of solid tumor, an area of normal white tumor, and an area of radionecrotic tissue with tumor cells, respectively. As shown in FIG. 5c, an unique fluorescence peak 504 at wavelength substantially around 500 nm is identified in the fluorescence spectrum curve 503, which is corresponding to a representative site or an area that contains radiation damaged brain tissues as confirmed by histology. Additionally, a secondary, yet an unique peak 510 at wavelength substantially around 500 nm is identified in curve 509, which is corresponding to a representative site that contains radionecrotic tissues with tumor cells. In comparison, curve 501 from a normal gray matter area, curve 505 from a solid tumor area and curve 507 from a normal white tumor area each has a fluorescence spectral peak 502, 506, or 508, correspondingly, substantially at around 460 nm. Peak 504 and peak 510 can be termed as a first peak, and peaks 502, 506, and 508 can be termed as a second peak.

Diffuse reflectance spectra for the five representative sites, shown as curves 511, 513, 515, 517, and 519 in FIG. 5d, respectively, display no distinct spectral variations that could be correlated to histological changes compatible with radiation damage.

According to the present invention, fluorescence spectroscopy is found to be more effective than diffuse reflectance spectroscopy in detecting radiation injuries of brain tissues. Diffuse reflectance spectroscopy, in principle, utilizes wavelength-dependence of tissue optics to interrogate microscopic-level structural variations in tissues. Because brain tissues are highly scattering [17, 18], most of the photons are scattered multiple times before they reemerge at the surface. Therefore, the intensity of diffuse reflectance is actually governed by the average scattering cross section of all scatters in tissues, instead of the scattering cross section of an individual scatter. Morphological changes in brain tissues resulting from radiation injuries, although obvious in histological examination, may not directly translate into a significant change in scattering properties of tissues. This limits the application of diffuse reflectance spectroscopy in detecting brain radiation injuries.

A clinic study over 136 sites of brain tissues from different patients was performed according to the present invention. Some of the general results are listed in Table 2. Overall, a good correlation was found between the existence of the unique fluorescence peak at wavelength around 500 nm emission and the histologically identifiable radiation injuries of brain tissues, from the 136 investigated sites, as shown in Table 2. Seventy six percent (19 out of 25) of the samples with histological characteristics of radiation injuries showed an unique fluorescence peak at 500 nm emission. In the 111 samples with no histological characteristics of radiation injuries, 11 were found to possess this unique fluorescence peak. The fluorescence peak at wavelength around 500 nm was present in tissues showing either pure radiation damage or a combination of radiation damage and tumor cells.

TABLE 2
Correlation between fluorescence spectral feature and histological findings

| | With Fluorescence | Without |
|---|---|---|
| | Peak at 500 nm | Fluorescence |
| | Emission | Peak at |
| | (“F_{500 nm} signature”) | 500 nm Emission |
| With Histological Characteristics | 19* | 6 |
| of Radiation Injury | | |
| Without Histological | 11 | 100 |
| Characteristics of Radiation | | |
| Injury | | |

*12 of these 19 samples contain tumor cells

The sensitivity of fluorescence spectroscopy in detecting early stages of brain radiation injuries may need further study. All patients except patient No. 1, who was transferred from an outside hospital and underwent urgent craniotomy during week 4 of that patient's external beam radiotherapy in order to prevent temporal lobe herniation, underwent radiation therapy weeks to months prior to the reoperation, as shown in Table 1. As a result, the initial responses of brain tissues to radiation may not be inferred from this study. As NAD(P)H is one of the prominent fluorophores responsible for tissue fluorescence and plays an important roles in tissue metabolism, one would expect that fluorescence spectroscopy could detect acute radiation response in brain tissues.

As indicated in column 2 of Table 2, 11 investigated samples with F_{500 nm} feature are found containing no histological features of radiation injuries. This discrepancy may be due to several possibilities. One is sampling error. The fiberoptic probe 340 used in the apparatus 300 has a small investigation area (i.e., less than 1 mm diameter); therefore, the biopsy samples might not be acquired from the exact tissue site at which optical investigation is performed. An alternative hypothesis is that fluorescence spectroscopy is more sensitive than histological examinations in terms of identifying radiation injuries of brain tissues. Unlike histology, fluorescence spectroscopy detects both morphological and biochemical changes in cells or tissues. Thus, it may be able to detect biochemical or molecular changes in cells resulting from radiation exposure prior to changes that are visible histologically. A third hypothesis is that neoplastic cells may overwhelm the histologic sample, obscuring underlying radiation damage. Four of the eleven cases where fluorescent peak at 500 nm emission are identified without histological changes came from a single patient with confluent areas of recurrent GBM. In these four sites, solid tumor may have prevented the pathologist from identifying histological characteristics of radiation damage.

Additionally, 6 samples with histological signatures of radiation injuries were observed not to possess fluorescence peak at 500 nm emission. This observation can be primarily attributed to the effects of superficial blood contamination, a thin layer of blood existing at the surface of the investigated tissue. Since oxyhemoglobin/hemoglobin (HbO₂/Hb) is a strong chromophore at the visible wavelength region, superficial blood contamination induces additional attenuation to the excitation as well as the emission light. As HbO₂/Hb has a prominent absorption peak at 420 nm, the primary fluorescence peak from brain tissues, approximately located at about 470 to 480 nm emission, would be shifted toward the longer wavelength region as the degree of superficial blood contamination increase. This, in turn, may reduce the feasibility of using this feature to differentiate radiation injuries from recurrent tumor. Despite the existence of an algorithm to subtract superficial blood contamination [19], that algorithm is not applicable in the situation of suspected radiation injury. Since this algorithm mathematically eliminates input from the wavelengths responsible for the blood contamination spectral interference, which have a large peak between 450 and 500 nm, it also subtracts the F_{500 nm} signature. Having recognized this as a major source of error, the inventors have altered their protocol for spectroscopy to include a saline rinse of the sample site and to surround the area with cottonoids to prevent blood rundown and spectral contamination. However, whether any hypothesis is correct or not does not in any way affect the validity and utility of the present invention.

The procedure most often used to obtain tissue is the stereotactic biopsy. Unfortunately, each biopsy taken with the stereotactic needle increases the risk of blood vessel injury and hematoma. Each specimen processed adds time and expense in both the operating room and the pathology suite. An adaptation to the current spectroscopy probe is being designed to aid in stereotactic biopsy. In this design, after the needle is passed and the inner trochar removed, a fiberoptic probe would be passed through the inner cannula to allow spectroscopic sampling of the adjacent brain tissue prior to biopsy. As optical spectroscopy has shown to reliably distinguish tumor from normal brain, the “optical biopsy” provided according to the present invention might prevent unnecessary tissue biopsy from areas with signal suggesting normal brain or radiation necrosis. If optical diagnosis may be achieved with a high degree of accuracy, it may allow fewer biopsies to be taken, thus reducing the time, expense and risk of these procedures.

Thus, among other things, according to the present invention, a novel in vivo fluorescence spectroscopy characteristic, fluorescence peak at 337 nm excitation and 500 nm emission, is identified in brain tissues with radiation injuries. This peak at 500 nm emission is distinct from the normal peak at 337 nm excitation and 460 nm emission observed in normal and tumor bearing brain tissues. Histopathologic analysis confirms either extensive radiation changes or frank radiation necrosis in the majority of the tissues with this 500 nm emission peak. Recognition of this peak in an area of enhancement on MRI appears to be diagnostic of radiation damage. Further development of this technology and discrimination algorithms may allow accurate “optical biopsy” which may allow the reduction of a portion of the risk and expense of repeated tissue sampling in the diagnosis of recurrent tumor within regions of radiation damage.

While there has been shown various embodiments of the present invention, it is to be understood that certain changes can be made in the form and arrangement of the elements of the apparatus and steps of the methods to practice the present invention as would be known to one skilled in the art without departing from the underlying scope of the invention as is particularly set forth in the Claims. For examples, while examples set forth above are related to practice the present invention in vivo, the present invention can be practiced in vitro as well. The coherent light can be emitted from a light source other than a laser light source. Furthermore, the embodiments described above are only intended to illustrate the principles of the present invention and are not intended to limit the claims to the disclosed elements.

List of References

[1] Kramer S, Lee K F: Complications of radiation therapy: the central nervous system, Semin Roentgenol, 1974, 9:75-83.

[2] Sheline G, Wara W, Smith V: An analysis of dose-effect relationship in the radiotherapy of malignant gliomas, Int J Radiat Oncol Biol Phys, 1980, 5:1725-1731.

[3] Hopewell J W: Late radiation damage to the central nervous system: a radiobiological interpretation, Neuropathol Appl Neurobiol, 1979, 5:329-43.

[4] van der Kogel A J: Radiation-induced damage in the central nervous system: an interpretation of target cell responses, Br J Cancer Suppl, 1986, 7:207-17.

[5] Valk P E, Dillon W P: Radiation injury of the brain, AJNR Am J Neuroradiol, 1991, 12:45-62.

[6] Saha G B, MacIntyre W J, Go R T: Radiopharmaceuticals for brain imaging, Semin Nucl Med, 1994, 24:324-49.

[7] Slizofski W J, Krishna L, Katsetos C D, et al: Thallium imaging for brain tumors with results measured by a semiquantitative index and correlated with histopathology, Cancer, 1994, 74:3190-7.

[8] Kahn D, Follett K A, Bushnell D L, et al: Diagnosis of recurrent brain tumor: value of 201Tl SPECT vs 18F-fluorodeoxyglucose PET, AJR Am J Roentgenol, 1994, 163:1459-65.

[9] Stokkel M, Stevens H, Taphoorn M, et al: Differentiation between recurrent brain tumour and post-radiation necrosis: the value of 201Tl SPET versus 18F-FDG PET using a dual-headed coincidence camera—a pilot study, Nucl Med Commun, 1999, 20:411-7.

[10] Tsui E Y, Chan J H, Leung T W, et al: Radionecrosis of the temporal lobe: dynamic susceptibility contrast MRI, Neuroradiology, 2000, 42:149-52.

[11] Kimura T, Sako K, Gotoh T, et al: In vivo single-voxel proton MR spectroscopy in brain lesions with ring-like enhancement, NMR Biomed, 2001, 14:339-49.

[12] Lin A, Bluml S, Mamelak A N: Efficacy of proton magnetic resonance spectroscopy in clinical decision making for patients with suspected malignant brain tumors, J Neurooncol, 1999, 45:69-81.

[13] Lin W-C, Toms S A, Johnson M, et al: In vivo brain tumor demarcation using optical spectroscopy, Photochem Photobio, 2001, 73:396-402.

[14] Ramanujam N: Fluorescence spectroscopy of neoplastic and non-neoplastic tissues, Neoplasia, 2000, 2:89-117.

[15] Sokolov K, Follen M, Richards-Kortum R: Optical spectroscopy for detection of neoplasia, Curr Opin Chem Biol, 2002, 6:651-8.

[16] Lin W-C, Toms S A, Motamedi M, et al: Brain tumor demarcation using optical spectroscopy; an in vitro study, J Biomedical Optics, 2000, 5:214-220.

[17] Eggert H R, Blazek V: Optical properties of normal human intracranial tissues in the spectral range of 400 to 2500 nm, Adv Exp Med Biol, 1993, 333:47-55.

[18] Svaasand L O, Ellingsen R: Optical properties of human brain, Photochem Photobiol, 1983, 38:293-299.

[19] Lin W-C, Toms SA, Jansen ED, et al: Intraoperative Application of Optical Spectroscopy in the Presence of Blood, IEEE Journal of Special Topics in Quantum Electronics :(in press), 2001.

What is claimed is:

1. A method for detecting radiation damage in an area of brain tissues, wherein the area of brain tissues has at least a first region containing brain tissues damaged from radiation exposure and a second region containing no brain tissues damaged from radiation exposure, comprising the steps of:
illuminating in vivo the area of brain tissues with a coherent light at an incident wavelength, λ₀, between 330 nm and 360 nm;
collecting electromagnetic emission returned from the illuminated brain tissues;
identifying a first peak of intensity of the collected electromagnetic emission at a first wavelength, λ₁, and a second peak of intensity of the collected electromagnetic emission at a second wavelength, λ₂, wherein λ₀, λ₁, and λ₂ satisfy the following relationship of λ₁>λ₂>λ₀; and
locating the first region containing brain tissues damaged from radiation exposure as the region of brain tissues where the first peak of intensity of the collected electromagnetic emission is corresponding to.

2. The method of claim 1, wherein the first region further comprises tumor.

3. The method of claim 1, wherein the second region comprises normal brain tissues.

4. The method of claim 3, wherein the second region further comprises tumor.

5. The method of claim 1, wherein the incident wavelength, λ₀, of the coherent light is substantially at around 337 nm.

6. The method of claim 1, wherein the coherent light is emitted from a laser light source.

7. The method of claim 1, wherein the first wavelength, λ₁, of the collected electromagnetic emission is substantially at around 500 nm.

8. The method of claim 7, wherein the second wavelength, λ₂, of the collected electromagnetic emission is substantially at around 460 nm.

9. An apparatus for detecting radiation damage in an area of brain tissues, wherein the area of brain tissues has at least a first region containing brain tissues damaged from radiation exposure and a second region containing no brain tissues damaged from radiation exposure, comprising:
means for illuminating in vivo the area of brain tissues with a coherent light at an incident wavelength, λ₀, between 330 nm and 360 nm;
means for collecting electromagnetic emission returned from the illuminated brain tissues;
means for identifying a first peak of intensity of the collected electromagnetic emission at a first wavelength, λ₁, and a second peak of intensity of the collected electromagnetic emission at a second wavelength, λ₂, wherein λ₀, λ₁, and λ₂ satisfy the following relationship of λ₁>λ₂>λ₀; and
means for locating the first region containing brain tissues damaged from radiation exposure as the region of brain tissues where the first peak of intensity of the collected electromagnetic emission is corresponding to.

10. The apparatus of claim 9, wherein the first region further comprises tumor.

11. The apparatus of claim 9, wherein the second region comprises normal brain tissues.

12. The apparatus of claim 11, wherein the second region further comprises tumor.

13. The apparatus of claim 9, wherein the incident wavelength, λ₀, of the coherent light is substantially at around 337 nm.

14. The apparatus of claim 9, wherein the means for illuminating comprises a laser light source.

15. The apparatus of claim 14, wherein the means for collecting comprises a fiber optical probe coupled with the laser light source so as to deliver in vivo the laser light to an area of brain tissues proximal a working end of the probe.

16. The apparatus of claim 15, wherein the means for collecting further comprises a spectroscope coupled with the fiber optical probe so as to receive from the working end of the probe, fluorescent light emitted from the area in response to illumination by the coherent light.

17. The apparatus of claim 16, wherein the means for identifying comprises a system controller operatively coupled with the spectroscope.

18. The apparatus of claim 9, wherein the first wavelength, λ₁, of the collected electromagnetic emission is substantially at around 500 nm.

19. The apparatus of claim 15, wherein the second wavelength, λ₂, of the collected electromagnetic emission is substantially at around 460 nm.

20. An apparatus for detecting radiation damage in an area of brain tissues, wherein the area of brain tissues has at least a first region containing brain tissues damaged from radiation exposure and a second region containing no brain tissues damaged from radiation exposure, comprising:
a laser light source emitting a coherent light with an incident wavelength, λ₀, between 330 nm and 360 nm;
a fiber optical probe coupled with the laser light source so as to deliver in vivo the laser light to an area of brain tissue proximal a working end of the probe;
a spectroscope coupled with the fiber optical probe so as to receive from the working end of the probe, fluorescent light emitted from the area in response to illumination by the coherent light;
a system controller operatively coupled with the spectroscope and configured to generate a spectrum having a first peak of intensity of the fluorescent light at a first wavelength, λ₁, and a second peak of intensity of the fluorescent light at a second wavelength, λ₂, wherein λ₀, λ₁, λ₂ satisfy the following relationship of λ₁>λ₂>λ₀.

21. The apparatus of claim 20, wherein the first region containing brain tissues damaged from radiation exposure is corresponding to the region of brain tissues where the first peak of intensity of the fluorescent light at the first wavelength λ₁ is emitted.

22. The apparatus of claim 21, wherein the first wavelength, λ₁, is substantially at around 500 nm.

23. A method for detecting radiation damage in an area of tissues associated with a living subject, wherein the area of tissues has at least a first region containing tissues damaged from radiation exposure and a second region containing no tissues damaged from radiation exposure, comprising the steps of:
illuminating the area of tissues with a coherent light at an incident wavelength, λ₀, between 330 nm and 360 nm;
collecting electromagnetic emission returned from the illuminated tissues;
identifying a first peak of intensity of the collected electromagnetic emission at a first wavelength, λ₁, and a second peak of intensity of the collected electromagnetic emission at a second wavelength, λ₂, wherein λ₀, λ₁, and λ₂ satisfy the following relationship of λ₁>λ₂>λ₀; and
locating the first region containing tissues damaged from radiation exposure as the region of tissues where the first peak of intensity of the collected electromagnetic emission is corresponding to.

24. The method of claim 23, wherein the first region further comprises tumor.

25. The method of claim 23, wherein the second region comprises normal tissues.

26. The method of claim 25, wherein the second region further comprises tumor.

27. The method of claim 23, wherein the incident wavelength, λ₀, of the coherent light is substantially at around 337 nm.

28. The method of claim 23, wherein the coherent light is emitted from a laser light source.

29. The method of claim 23, wherein the first wavelength, λ₁, of the collected electromagnetic emission is substantially at around 500 nm.

30. The method of claim 29, wherein the second wavelength, λ₂, of the collected electromagnetic emission is substantially at around 460 nm.

31. The method of claim 23, wherein the living subject is a human being.

32. The method of claim 23, wherein the living subject is an animal.

33. An apparatus for detecting radiation damage in an area of tissues associated with a living subject, wherein the area of tissues has at least a first region containing tissues damaged from radiation exposure and a second region containing no tissues damaged from radiation exposure, comprising the steps of:
means for illuminating the area of tissues with a coherent light at an incident wavelength, λ₀, between 330 nm and 360 nm;
means for collecting electromagnetic emission returned from the illuminated tissues;
means for identifying a first peak of intensity of the collected electromagnetic emission at a first wavelength, λ₁, and a second peak of intensity of the collected electromagnetic emission at a second wavelength, λ₂, wherein λ₀, λ₁, and λ₂ satisfy the following relationship of λ₁>λ₂>λ₀; and
means for locating the first region containing tissues damaged from radiation exposure as the region of tissues where the first peak of intensity of the collected electromagnetic emission is corresponding to.

34. The apparatus of claim 33, wherein the first region further comprises tumor.

35. The apparatus of claim 33, wherein the second region comprises normal tissues.

36. The apparatus of claim 35, wherein the second region further comprises tumor.

37. The apparatus of claim 33, wherein the incident wavelength, λ₀, of the coherent light is substantially at around 337 nm.

38. The apparatus of claim 37, wherein the means for illuminating comprises a laser light source.

39. The apparatus of claim 38, wherein the means for collecting comprises a fiber optical probe coupled with the laser light source so as to deliver in vivo the laser light to an area of tissues proximal a working end of the probe.

40. The apparatus of claim 39, wherein the means for collecting further comprises a spectroscope coupled with the fiber optical probe so as to receive from the working end of the probe, fluorescent light emitted from the area in response to illumination by the coherent light.

41. The apparatus of claim 40, wherein the means for identifying comprises a system controller operatively coupled with the spectroscope.

42. The apparatus of claim 33, wherein the first wavelength, λ₁, of the collected electromagnetic emission is substantially at around 500 nm.

43. The apparatus of claim 42, wherein the second wavelength, λ₂, of the collected electromagnetic emission is substantially at around 460 nm.

44. The apparatus of claim 33, wherein the living subject is a human being.

45. The apparatus of claim 33, wherein the living subject is an animal.
